(19) United States
(12) Patent Application Publication
(10) Pub. No.: US 2025/0369965 A1
(43) Pub. Date: Dec. 4, 2025

(54) FORMULATION, A KIT, AND METHOD FOR FOOD ALLERGEN DETECTION

(71) Applicant: LEO Verification Systems, Inc., Powell, WY (US)

(72) Inventors: Wen-Hao CHEN, Powell, WY (US); Yuh-Cherng CHU, Powell, WY (US); Jill SMITH, Powell, WY (US); Seaton SMITH, Powell, WY (US); Hui-Yin HUANG, Powell, WY (US)

(21) Appl. No.: 19/220,060

(22) Filed: May 27, 2025

Related U.S. Application Data
(60) Provisional application No. 63/652,467, filed on May 28, 2024.

(51) Int. Cl. G01N 33/543; C07D 233/58; G16H 30/20

(52) U.S. Cl. CPC G01N 33/54388; C07D 233/58; G16H 30/20

(57) ABSTRACT

The present disclosure provides a formulation for extracting a water-insoluble protein, as well as a kit and a method using the formulation to rapidly detect gluten allergen. The formulation includes a Tris buffer and an ionic liquid.

### CROSS-REFERENCE TO RELATED APPLICATIONS

This application claims priority to U.S. Provisional Patent Application No. 63/652,467, filed on May 28, 2024. The entirety of the above-mentioned patent application is hereby incorporated by reference herein and made a part of this specification.

### BACKGROUND

### 1. Technical Field

The present disclosure relates to a formulation for extracting a water-insoluble protein, along with a kit and a system utilizing the formulation for the rapid detection of gluten allergens.

### 2. Description of Associated Art

Estimates from the Centers for Disease Control and Prevention (CDC) reveal that more than 50 million people in America experience allergies annually. According to the CDC's National Health Interview Survey, 6.2% of adults and 5.8% of children have food allergies, and approximately US$25 billion is spent on food allergen treatment each year in the United States. Even trace amounts of allergens can trigger acute anaphylaxis, a potentially life-threatening hypersensitivity reaction requiring epinephrine injections. The Food Allergen Labeling and Consumer Protection Act (FALCPA) mandates food labeling to inform consumers about allergenic substances in products. However, mislabeling and cross-contamination in manufacturing continue to pose regulatory challenges. Furthermore, the FALCPA covers only packaged foods, not those served in restaurants. The ability to rapidly test foods for common allergens also remains a major unmet need.

Wheat is a major food source worldwide, but it is also a common food allergen. Celiac disease is a chronic immune-mediated enteropathy triggered by exposure to dietary gluten in genetically predisposed individuals; it can only be treated through strict gluten avoidance. Therefore, rapid gluten detection is crucial for protecting the health of patients.

Researchers have used various analytical techniques and devices to analyze gluten levels in processed and unprocessed foods; these techniques and devices include polymerase chain reaction (PCR), liquid chromatography-tandem mass spectrometry (LC-MS/MS), microarrays, immunosensors, aptasensors, matrix-assisted laser desorption/ionization time-of-flight mass spectrometry (MALDI-TOF MS), near-infrared (NIR) spectroscopy, and electrochemical sensing. In recent years, enzyme-linked immunosorbent assay (ELISA) and lateral flow assay techniques have been commonly used to comprehensively analyze gliadin content.

However, gluten contains two primary water-insoluble proteins: gliadin and glutenin. Gliadin, a significant contributor to celiac disease, presents challenges for sample pretreatment due to its insolubility, which adversely affects the accuracy and sensitivity of detection systems. Therefore, rapid sample processing is a critical problem in gliadin detection. Thus, there is an urgent need in the field to develop a formulation capable of extracting the water-insoluble protein such as gliadin, as well as a kit, method and/or system designed for the rapid detection of gluten in food or other materials.

### SUMMARY

Given the disadvantages of the prior art described above, the present disclosure provides a formulation that can rapidly extract water-insoluble proteins such as gliadin and kit using the formulation to detect gluten. The present disclosure also provides an integrated food allergy and microorganism sensor (iFAMs) system. The system enables gliadin extraction and detection in under 3 min with high sensitivity (0.04 mg/kg for gliadin, lower than the regulatory limit of 20 mg/kg). Users can easily measure gluten concentrations in samples and quantify gliadin levels using an electronic device (including a smartphone-based image assay app). The compact size and user-friendly design of the iFAMs system render it suitable for not only consumers but also clinicians, food industries, and regulators to enhance food safety.

For the purpose above, the present disclosure provides a formulation for extracting a water-insoluble protein, comprising a Tris buffer and an ionic liquid.

In one embodiment of the formulation of the present disclosure, the ionic liquid is comprised in the formulation at a concentration of 0.05-1.5 wt %, based on the total weight of the formulation.

In one embodiment of the formulation of the present disclosure, the ionic liquid is an imidazolium-based ionic liquid comprising an imidazolium cation and an organic or inorganic anion, wherein the imidazolium cation has a structure of formula (I):

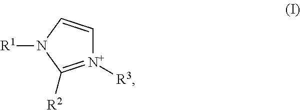

wherein R¹ is H or methyl, R² is H or methyl, and R³ is absent or an alkyl group having 3 to 12 carbon atoms,
and wherein the anion is selected from the group consisting of methanesulfonate (MSO), Cl⁻, F⁻, NO₃⁻, HSO₄⁻ and H₂PO₄⁻.

In one embodiment of the formulation of the present disclosure, the imidazolium cation is pentyl dimethyl imidazolium, heptyl dimethyl imidazolium, nonyl dimethyl imidazolium or dodecyl dimethyl imidazolium, and the anion is methanesulfonate, NO₃⁻, HSO₄⁻ or H₂PO₄⁻.

In one embodiment of the formulation of the present disclosure, the water-insoluble protein is gliadin.

In one embodiment of the formulation of the present disclosure, the formulation has a pH value ranges from 1 to 6.

The present disclosure further provides a kit for detecting gluten, comprising:
a lateral flow chip, which comprises
   a backing card;
   an assay membrane disposed on the baking card, wherein the assay membrane has a detecting area with a first anti-gliadin antibody immobilized for forming a test line, and a secondary antibody against a AuNP-conjugated antibody immobilized for forming a control line, wherein the AuNP-conjugated antibody is formed by conjugating a second anti-gliadin antibody and an Au nanoparticle, and the control line being arranged staggered position relative to the test line;
   a conjugate pad disposed upstream of the assay membrane, wherein the conjugate pad is adjacent to or partially covers the assay membrane, and at least part of the conjugate pad is coated with the AuNP-conjugated antibody;
   an absorbent pad disposed downstream of the assay membrane, wherein the absorbent pad is adjacent to or partially covers the assay membrane, with a space between the conjugate pad and the absorbent pad to expose the detecting area of the assay membrane; and
   a sample pad disposed upstream of the conjugate pad, wherein the sample pad is adjacent to or partially covers the conjugate pad; and
      a container, which comprises the aforementioned formulation.

In one embodiment of the kit of the present disclosure, a material forming the assay membrane is selected from at least one of the group consisting of nitrocellulose, polyvinylidene difluoride (PVDF) and cellulose acetate.

In one embodiment of the kit of the present disclosure, the first anti-gliadin antibody and the second anti-gliadin antibody are different from each other and are independently selected from monoclonal antibody, polyclonal antibody and recombinant antibody.

In one embodiment of the kit of the present disclosure, each of the first anti-gliadin antibody and the second anti-gliadin antibody is selected from mouse anti-gliadin antibody, rabbit anti-gliadin antibody and recombinant human anti-gliadin antibody.

In one embodiment of the kit of the present disclosure, the kit further comprises an identification code.

In one embodiment of the kit of the present disclosure, the identification code is a 1D Barcode and/or a 2D Barcode.

In one embodiment of the kit of the present disclosure, the kit further comprises a color reference mark on the lateral flow chip.

In one embodiment of the kit of the present disclosure, the kit further contains a sampling tool for sampling the material to be tested.

In one embodiment of the kit of the present disclosure, the kit further contains an operation instruction.

The present disclosure further provides a method for detecting a food allergen, comprising: receiving an image via a data transmission module of a cloud server, wherein the image includes the detecting area, the identification code, and the color reference mark of the kit of the present disclosure, and wherein the image is captured by an electronic device and transmitted to the cloud server; normalizing and calculating a gliadin concentration via the cloud server by utilizing a standard line of gliadin concentration stored in the cloud server based on the color intensity of the detecting area in the image; and transmitting the calculated gliadin content including the gliadin concentration from the cloud server back to the electronic device via the data transmission module.

In one embodiment of the method of the present disclosure, the image includes a test line image and a control line image formed in the detecting area, and the method further comprising: calibrating a color of the color reference mark and simultaneously calibrating colors of the test line image and the control line image by a color calibration module of the cloud server; and converting the calibrated colors of the test line image and the control line image to 8-bit grayscale to obtain a color intensity of the test line image and the control line image by the color calibration module of the cloud server.

In one embodiment of the method of the present disclosure, the gliadin content with timestamp and location is stored in a database.

In one embodiment of the method of the present disclosure, the database is a cloud database.

In one embodiment of the method of the present disclosure, the database storing a plurality of standard lines for multiple different kit product batches, and the method further comprising: determining, via the cloud server, the corresponding product batch based on the received identification code; retrieving, via the cloud server, the standard lines associated with the determined product batch from the database storing standard lines for multiple different kit product batches; and normalizing, via the cloud server, the color intensity to the gliadin concentration based on the retrieved standard line.

The present disclosure further provides a system for detecting a food allergen, comprising:
a cloud server comprising:
   a data transmission module configured to receive an image containing the detecting area, the identification code, and the color reference mark of the kit of the present disclosure; and
   a processing unit configured to normalize and calculate a gliadin concentration by utilizing a standard line of gliadin concentration stored in the cloud server based on the color intensity of the detecting area in the received image;
      wherein the data transmission module is configured to transmit the calculated gliadin content, including the gliadin concentration, from the cloud server to an external device.

In one embodiment of the system of the present disclosure, the system further comprises an electronic device configured to capture an image of the detecting area, the identification code, and the color reference mark of the kit; and to transmit the captured image to the cloud server.

In one embodiment of the system of the present disclosure, the image includes a test line image and a control line image formed in the detecting area, and the cloud server further comprises a color calibration module configured to calibrate the color of the color reference mark and simultaneously calibrate the colors of the test line image and the control line image; and to convert the calibrated colors of the test line image and the control line image to 8-bit grayscale to obtain the color intensity of the test line image and the control line image.

In one embodiment of the system of the present disclosure, the cloud server is configured to store the gliadin content with a timestamp and location in a database.

In one embodiment of the system of the present disclosure, the database is a cloud database storing a plurality of standard lines for multiple different kit product batches.

In one embodiment of the system of the present disclosure, the processing unit is configured to determine the corresponding product batch based on the received identification code and to receive at least one color intensity.

In one embodiment of the system of the present disclosure, the cloud server further comprises: a retrieval module configured to retrieve the standard lines associated with the determined product batch from the database storing standard lines for multiple different kit product batches; and a normalization unit configured to normalize the received color intensity to the gliadin concentration based on the retrieved standard line.

According to the present disclosure, a computer program product is provided and utilizes the app, firmware, or cloud technology of the aforementioned system to execute the aforementioned method, and the computer program product can automatically store the gliadin content with timestamp and location in the cloud server.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present disclosure can be more fully understood by reading the following descriptions of the embodiments, with reference made to the accompanying drawings.

FIG. 1 shows the exploded view of the lateral flow chip of the present disclosure.

FIG. 2 shows the components comprised in the kit of the present disclosure, which comprises a lateral flow chip and a container comprising the formulation of the present disclosure.

FIGS. 3A to 3D show the appearance of AuNPs with or without antibodies conjugated therewith (3A); the TEM image of AuNPs (3B); the UV-VIS spectra of AuNPs with (red line) or without (black line) antibody conjugated therewith (3C); and size distribution of AuNPs (3D).

FIG. 4 is a schematic diagram showing an integrated food allergy and microorganism sensor (iFAMs) system.

FIG. 5A shows an image of a test line and a control line is converted to 8-bit grayscale.

FIG. 5B shows samples with varying doses of gliadin that were analyzed by the iFAMs system and a response line that was generated.

FIG. 6A shows the intra-assay variations of the iFAMs system.

FIG. 6B shows iFAMs gluten test (left to right: 0.01, 0.1, 1 10 and 20 ppm) results.

FIG. 6C shows the color intensity of the test line, which increased with the gluten concentration.

FIG. 6D shows the specificity of the iFAMs for different flour samples, including wheat, oat, corn, quinoa, rice, chickpea, chestnut and almond.

FIGS. 7A to 7D show the extraction performances of different buffers (7A); extraction performances of PBS buffers with or without ionic liquid at different pH values (7B); extraction performances of different buffers with or without ionic liquid (7C); and time dependency of extraction performances of Tris buffer with (black line) or without (red line) ionic liquid (7D).

FIG. 8A and FIG. 8C are flow charts showing steps of a method for detecting a food allergen according to the present disclosure.

### DETAILED DESCRIPTION OF THE PREFERRED EMBODIMENTS

The following examples are used for illustrating the present disclosure. A person skilled in the art can easily conceive the other advantages and effects of the present disclosure, based on the disclosure of the specification. The present disclosure can also be implemented or applied as described in different examples. It is possible to modify or alter the following examples for carrying out this disclosure without contravening its scope, for different aspects and applications.

Wheat is a major global food source, but it contains gluten, a food allergen that induces immune responses in individuals with celiac disease and nonceliac gluten sensitivity (NCGS). Moreover, wheat gluten includes gliadin; the primary toxic component of gliadin is a 33-mer peptide from alpha-2-gliadin, which contains proline and glutamine amino acid residues. This peptide is often described as the most critical celiac disease immunogenic sequence in gliadin.

Celiac disease is an autoimmune illness caused by an immune reaction to gluten consumption. This chronic immune-mediated enteropathy occurs in genetically predisposed individuals upon exposure to dietary gluten. In individuals with celiac disease, gluten ingestion activates both innate and adaptive immune responses. These structural changes lead to functional impairment of the intestinal mucosa, resulting in symptoms caused by nutrient malabsorption.

Currently, the most common treatment for celiac disease involves a strict, lifelong gluten-free diet and/or the consumption of foods with a “gluten-free” label. According to the Codex Standard 118-1979 (adopted by the US Food and Drug Administration, FR Doc. 2013-18813) and European Commission Regulation (EC 41/2009), gluten levels in designated gluten-free foodstuffs should not exceed 20 parts per million (ppm).

Gluten comprises several proteins, including alpha, gamma, and omega gliadin, as well as high and low-molecular-weight glutenins. Gliadin is the primary contributor to celiac disease and belongs to a family of water-insoluble proteins. Conventional pretreatment processes for extracting gliadin from heat-processed or diluted samples (1:50) after alcohol extraction are time-consuming. Furthermore, diluting samples after alcohol extraction often requires skilled personnel. Regardless of the pretreatment process, conventional gliadin detection methods necessitate specialized equipment to measure gliadin concentrations, making them less user-friendly for consumers. Therefore, efficient gliadin extraction remains a considerable challenge in the art.

The present disclosure is directed to a formulation for extracting a water-insoluble protein, comprising a Tris buffer and an ionic liquid. Examples of the water-insoluble protein that are suitable for being extracted by the formulation of the present disclosure include, but not limited to, gliadin and glutenin. In one embodiment, the formulation of the present disclosure is used for extracting gliadin.

As used in the present disclosure, the term “Tris buffer” refers to buffer solutions comprising tris(hydroxymethyl) aminomethane (Tris). In one embodiment, Tris buffer is prepared by dissolving Tris base in distilled water and adjusting the solution to desired pH by using HCl. In another embodiment, Tris buffer can further contain additional ingredients such as glycine, acetate EDTA, borate EDTA and 6-aminocaproic acid (EACA).

As used in the present disclosure, the term “ionic liquid” refers to a class of non-molecular compounds that are composed solely of ions (including cations and anions). Examples of cations in the ionic liquid include, but not limited to, imidazolium, pyridinium, quaternary ammonium and quaternary phosphonium. Furthermore, anions in the ionic liquid can be organic or inorganic anion including, but not limited to, halogen (such as Cl⁻ and F⁻), triflate, tetrafluoroborate, hexafluorophosphate, methanesulfonate (MSO), NO₃⁻, HSO₄⁻ and H₂PO₄⁻.

In one embodiment, the ionic liquid used in the present disclosure contains imidazolium cation, which has a structure of formula (I):

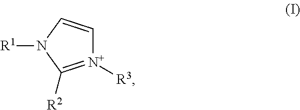

wherein R¹ is H or methyl, R² is H or methyl, and R³ is absent or an alkyl group having 3 to 12 carbon atoms. In one embodiment, a range of the number of carbon atoms in the alkyl group of R³ can extend from a lower limit to an upper limit, for example, 3, 4, 5, 6, 7, 8, 9, 10, 11 or 12. In a preferred embodiment, R³ is an alkyl group having 5, 7, 9 or 12 carbon atoms.

In some embodiments, the ionic liquid used in the present disclosure comprises imidazolium cations such as pentyl dimethyl imidazolium, heptyl dimethyl imidazolium, nonyl dimethyl imidazolium and dodecyl dimethyl imidazolium, and anions such as methanesulfonate, NO₃⁻, HSO₄⁻ and H₂PO₄⁻.

The amount of the ionic liquid comprised in the formulation is not limited, as long as the ionic liquid can be fully and homogeneously mixed with the Tris buffer. In one embodiment, the ionic liquid is comprised in the formulation at a concentration of 0.01-10 wt %, based on the total weight of the formulation. In a preferred embodiment, the ionic liquid is comprised in the formulation at a concentration of 0.05-1.5 wt %, based on the total weight of the formulation. For example, the ionic liquid is comprised in the formulation at a concentration of 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.05, 1.1, 1.15, 1.2, 1.25, 1.3, 1.35, 1.4, 1.45 or 1.5 wt %, based on the total weight of the formulation. More preferably, the ionic liquid is comprised in the formulation at a concentration of 0.5-1 wt %, based on the total weight of the formulation.

The pH value of the formulation of the present disclosure can be adjusted according to various factors such as the water-insoluble protein to be extracted, the type of the ionic liquid being used and the concentration of the ionic liquid. In one embodiment, the formulation of the present disclosure has a pH value ranges from 1 to 6. For example, the formulation of the present disclosure has a pH value of 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9 or 6.0. Preferably, the ionic liquid has a pH value ranges from 5 to 6.

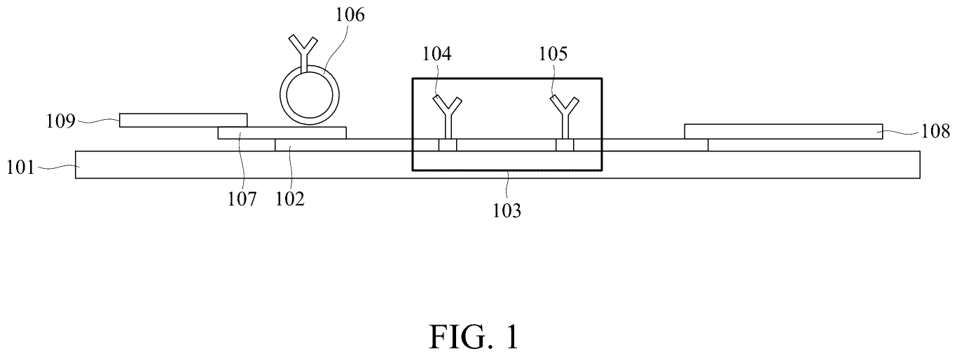

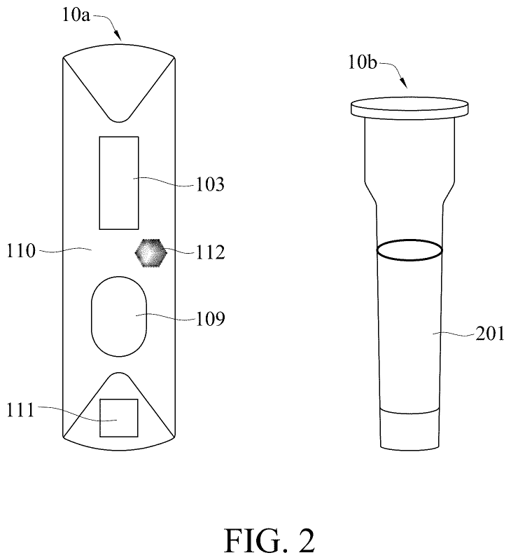

As shown in FIGS. 1 and 2, the present disclosure also directs to a kit 10 for detecting gluten, comprising:
a lateral flow chip 10a, which comprises
   a backing card 101;
   an assay membrane 102 disposed on the baking card 101, wherein the assay membrane 102 has a detecting area 103, in which a first anti-gliadin antibody is immobilized thereon to constitute a test line 104, and a secondary antibody against a AuNP-conjugated antibody 106 is immobilized thereon to constitute a control line 105, wherein the AuNP-conjugated antibody 106 is formed by conjugating a second anti-gliadin antibody and an Au nanoparticle, and wherein the control line 105 is arranged staggered position relative to the test line 104;
   a conjugate pad 107 disposed upstream of the assay membrane 102, wherein the conjugate pad 107 is adjacent to or partially covers and contacts the assay membrane 102, and at least part of the conjugate pad 107 is coated with the AuNP-conjugated antibody 106;
   an absorbent pad 108 disposed downstream of the assay membrane 102, wherein the absorbent pad 108 is adjacent to or partially covers and contacts the assay membrane 102, with a space between the conjugate pad 107 and the absorbent pad 108 to expose the detecting area 103 of the assay membrane 102; and
   a sample pad 109 disposed upstream of the conjugate pad 107, wherein the sample pad 109 is adjacent to or partially covers and contacts the conjugate pad 107; and
a container 10b, which comprises the aforementioned formulation 201 of the present disclosure.

The kit 10 of present disclosure can effectively extract gliadin from unprocessed and heat-processed foods and then measures the gliadin concentration through a lateral flow test within 2 min without additional equipment. This sensor is suitable for use by untrained individuals of all ages, even in remote areas lacking advanced medical laboratories.

In the present disclosure, the lateral flow chip 10a or components thereof can be obtained commercially or be prepared by methods known in the art. For example, the lateral flow chip can be prepared by providing a backing card 101 (usually is a plastic sheet) and forming an assay membrane 102, a conjugate pad 107, an absorbent pad 108 and a sample pad 109 thereon. After those components are combined, a first antibody against gliadin is dispensed onto the assay membrane 102 to constitute a test line 104; and a second antibody against the same target molecular is conjugated to an Au nanoparticle to form a AuNP-conjugated antibody 106 and then coated onto the conjugate pad 107. A secondary antibody against the AuNP-conjugated antibody is then dispensed onto the assay membrane 102 to constitute a control line 105. The control line 105 is staggered with the test line 104, and the distance between the control line 105 and the test line 104 can be determined by a person skilled in the art and can be, for example, about 1 mm to about 10 mm, preferably, 4 mm, 5 mm, 6 mm, 7 mm or 8 mm. In this disclosure, the area on the assay membrane 102 encompasses the test line 104 and the control line 105 is called detecting area 103. Methods for combining the aforementioned components and dispensing the antibodies are well known in the art.

In one embodiment, the assay membrane 102 used in the lateral flow chip 10a can be composed of any material that is suitable for immobilizing antibodies and allows the sample fluid flowing through. For example, a material forming the assay membrane is selected from at least one of the group consisting of nitrocellulose, polyvinylidene difluoride (PVDF) and cellulose acetate.

In one embodiment, the conjugate pad 107 is disposed upstream of the assay membrane 102, wherein the conjugate pad 107 is adjacent to or partially covers and contacts the assay membrane 102. At least part of the conjugate pad 107 is exposed, and thus can be coated with the AuNP-conjugated antibody 106 (i.e., the conjugate formed by the second antibody against the gliadin and Au nanoparticle). The material for forming the conjugate pad 107 is not limited, as long as it can release the AuNP-conjugated antibody 106 when the sample fluid flowing through. Examples of the materials used for forming the conjugate pad 107 include, but are not limit to, cellulose, glass and plastic.

In one embodiment, the absorbent pad 108 is disposed downstream of the assay membrane 102, wherein the absorbent pad 108 is adjacent to or partially covers and contacts the assay membrane 102, provided that there is a space between the conjugate pad 107 and the absorbent pad 108 to expose the detecting area 103 of the assay membrane 102. The absorbent pad 108 is used for increasing the force caused by capillary action, and thereby accelerating the sample fluid to flow from the sample pad 109 to the absorbent pad 108.

In one embodiment, the sample pad 109 is disposed upstream of the conjugate pad 107, wherein the sample pad 109 is adjacent to or partially covers and contacts the conjugate pad 107. The sample pad 109 is used for absorbing the sample fluid being dripped onto the chip. Materials that are commonly used as sample pads 109 include, but are not limited to, cellulose fiber filters and woven meshes.

In some embodiments, the first anti-gliadin antibody and the second anti-gliadin antibody can be same or different from each other. In one embodiment, the first anti-gliadin antibody and the second anti-gliadin antibody are different from each other and are independently selected from monoclonal antibody, polyclonal antibody and recombinant antibody. In one embodiment, each of the first anti-gliadin antibody and the second anti-gliadin antibody is selected from mouse anti-gliadin antibody, rabbit anti-gliadin antibody and recombinant human anti-gliadin antibody. Those antibodies can be obtained commercially or obtained by immunizing animals such as mouse, rabbit, goat, guinea pig, hamster, horse, monkey and human. Examples of the antibodies that can be used in the present disclosure are shown in the following Table 1:

TABLE 1

| Antibodies | Catalog No. | Agency | State |
|---|---|---|---|
| Mouse Anti-Gliadin IgG | 7148 | Chondrex Inc. | Monoclonal |
| Rabbit Anti-Gliadin IgG | G9144 | Merk | Polyclonal |
| Gliadin peptide antibody | 14D5 | Enzo life science | monoclonal |
| Gliadin Antibody | 4F3 | Invirtogen | Monoclonal |
| Gliadin Antibody | clone 6F113 | LifeSpan | Monoclonal |
| | | Biosciences | |
| Gliadin Antibody | LS-C212222 | LifeSpan | Monoclonal |
| | | Biosciences | |
| Rabbit Anti-Gliadin | G9144 | MilliporeSigma | Monoclonal |
| Mouse Anti-Gliadin | clone GLD7 | Merk | Monoclonal |
| Wheat gliadin antibody | CAC07296 | Biomatik | Polyclonal |
| Recombinant Human | MHH-397 | Creative Biolabs | Recombinant |
| Anti-Gliadin Antibody | | | |
| Mouse Anti-Gliadin | CABT- | Creative | monoclonal |
| antibody | L2556D | Diagnostics | |
| Gliadin Antibody | abx110677 | Abbexa Ltd | Polyclonal |
| Wheat Gliadin Antibody | orb243824 | Biorbyt | Polyclonal |

In one embodiment, the first anti-gliadin antibody can be selected from the group consisting of Mouse Anti-Gliadin IgG (7148, Chondrex Inc), Gliadin peptide antibody (14D5, Enzo life science) and Gliadin antibody (LS-C21222, lifespan Biosciences).

In one embodiment, the second anti-gliadin antibody can be selected from the group consisting of Rabbir Anti-gliadin IgG (G9144, Merk), Rabbit Anti-gliadin (G9144, Millipore Sigma) and Mouse anti-gliadin antibody (CABT-L2556D, Creative Diagnostics).

In one embodiment, the secondary antibody can be obtained commercially or designed in the laboratory. The secondary antibodies suitable for being used in the present disclosure are well known in the art, and can be chose by a person skilled in the art based on the second antibody used in the chip.

In one embodiment, the first anti-gliadin antibody and the secondary antibody can be dispensed onto the assay membrane at a concentration of about 1 mg/ml, and at an amount of 20-30 μl. In one embodiment, the second anti-gliadin antibody is conjugated with the AuNP in a solution, wherein the solution is at a pH value of about 8, and the second anti-gliadin antibody presented in the solution at a concentration of 0.3-1.2 μg/ml, such as 0.3, 0.4, 0.5, 0.6, 0.7, 0.8, 0.9, 1.0, 1.1 and 1.2 μg/ml.

In one embodiment, the container 10b comprising the formulation 201 of the present disclosure can be any container that is capable of accommodating the formulation 201 of the present disclosure and would not react with the ingredients in the formulation 201. Examples of the container 10b include, but are not limited to, bottle, tube, vial, ampule, cartridge and syringe.

In one embodiment, the lateral flow chip 10a can further comprise a housing 110 that can accommodate the combined backing card 101, assay membrane 102, conjugate pad 107, absorbent pad 108 and sample pad 109. The most commonly used housing 110 is plastic housing, but is not limited thereto. The housing 110 comprises at least one opening to expose the sample pad 109 and at least one window to show the detecting area 103.

In one embodiment, the kit 10 can further comprise an identification code 111 to identify the type, batch, serial number or other information of the kit. The identification code 111 can be disposed on any place, as long as the identification code 111 can be clearly read or photographed. For example, the identification code 111 is disposed on the surfaces of the chip (such as the top surface of the housing 110), on the container 10b, on the inner packing or the outer packing of the kit 10 or on a separated information sheet or operation instruction. In one embodiment, the mark can be 1D Barcode and/or a 2D Barcode.

In one embodiment, the kit further comprises a color reference mark 112 on the lateral flow chip 10a. For example, the color reference mark 112 can be a mark with monocolor or multicolor. This mark can be printed on the housing 110 of the lateral flow chip 10a and thus can be used to calibrate the colors of the test line and the control line.

In one embodiment, the kit 10 further contains a sampling tool for sampling the material to be tested, for example, food. In one preferred embodiment, the material to be tested is food served in restaurants. The sampling tool can be formed by any material (e.g, plastic, wood, metal or glass) and in any shape (e.g., rod-shaped, strip-shaped, cone-shaped or threadlike), as long as it can be used to obtain part of the material to be tested and put into the formulation 201 contained in the container 10b to form the sample fluid.

In one embodiment, the kit further contains an operation instruction. The operation instruction can be presented in any form, such as a printed copy, a web page, an image and a video. In one embodiment, the operation instruction can be obtained by the user from the web page or App provided by the supplier.

The kit 10 of the present disclosure is easy to use and suitable for use by untrained individuals of all ages. Specifically, users can simply detect the gliadin by sampling the material to be tested and extracted the sample with the formulation 201 of the present disclosure contained in the container 10b to obtain a sample fluid, and then dropping few drops of the obtained sample fluid to the sample pad 109 of the chip 10a. If gliadin is present in the sample fluid, it binds to the AuNP-conjugated antibody 106 coated on the conjugate pad 107. This complex is then captured by the first anti-gliadin antibody immobilized on the test line 104. Excess AuNP-conjugated antibody is captured by the secondary antibody immobilized on the control line 105. The appearance of two red lines on the chip indicates a positive result, and the appearance of only the control line signifies a negative result. The test result was observed in the result window after 2 min.

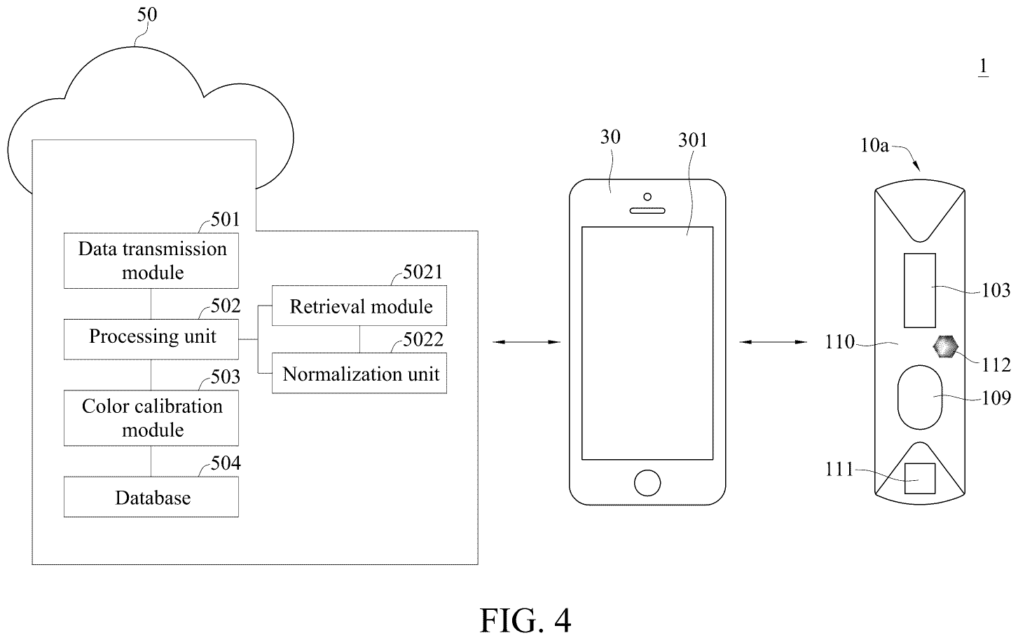

FIG. 4 is a schematic diagram showing the integrated food allergy and microorganism sensor (iFAMs) system. For example, the system 1 is use for detecting a food allergen, comprising: a cloud server 50 comprising: a data transmission module 501 configured to receive an image containing the detecting area, the identification code, and the color reference mark of the kit of the present disclosure; and a processing unit 502 configured to normalize and calculate a gliadin concentration by utilizing a standard line of gliadin concentration stored in the cloud server 50 based on the color intensity of the detecting area in the received image; wherein the data transmission module 501 is configured to transmit the calculated gliadin content, including the gliadin concentration, from the cloud server 50 to an external device. In one embodiment, the system 1 further comprises an electronic device 30 configured to capture an image of the detecting area, the identification code, and the color reference mark of the kit; and to transmit the captured image to the cloud server 50, and wherein the image includes a test line image and a control line image formed in the detecting area. For example, the electronic device 30 may be a smartphone, a laptop, a tablet, or a desktop computer, or it may be another device capable of capturing images and transmitting or receiving data.

Specifically, the computational operations of the cloud server 50 are executed by one or more processing units, which may be implemented as Central Processing Units (CPUs).

In one embodiment, the cloud server 50 further comprises a color calibration module 503 configured to calibrate the color of the color reference mark and simultaneously calibrate the colors of the test line image and the control line image; and to convert the calibrated colors of the test line image and the control line image to 8-bit grayscale to obtain the color intensity of the test line image and the control line image.

In one embodiment, the cloud server 50 is configured to store the gliadin content with a timestamp and location in a database 504. For example, the database 504 is a cloud database storing a plurality of standard lines for multiple different kit product batches. Each standard line can serve as an index for the corresponding color intensity and gliadin concentration. The database 504 may be implemented using various storage solutions, including high-capacity Hard Disk Drives (HDDs), Solid State Drives (SSDs), or distributed storage systems, managed by storage controllers to ensure efficient data storage and retrieval. For large-scale deployments, database server hardware, comprising multi-core processing units and substantial memory, may be utilized.

In one embodiment, wherein the processing unit 502 is configured to determine the corresponding product batch based on the received identification code and to receive at least one color intensity; and the cloud server 50 or the processing unit 502 further comprises: a retrieval module 5021 configured to retrieve the standard lines associated with the determined product batch from the database 504 storing standard lines for multiple different kit product batches; and a normalization unit 5022 configured to normalize the received color intensity to the gliadin concentration based on the retrieved standard line.

Specifically, the processing unit 502 is responsible for orchestrating the calibration process. Upon receiving an identification code from a kit of an actual product, transmitted via a data transmission module 501, the processing unit 502 initiates a data retrieval process. The data transmission module 501, facilitating communication with external devices and networks, can be realized through Network Interface Cards (NICs) (for internet-based communication), Bus Interfaces (like PCI-e for internal communication within the server), and potentially Serial/Parallel Communication Interfaces (for direct connections to specialized hardware).

The processing unit 502 then employs a retrieval module 5021 to access the database 504. The retrieval module 5021 manages the process of locating and extracting the standard line corresponding to the product batch identified by the received identification code. In distributed database scenarios, the retrieval module 5021 might utilize Network Interface Cards (NICs) to communicate with remote database nodes.

The processing unit 502 further receives calibrated color intensity data, again facilitated by the data transmission module 501. This color intensity data is often derived from an image captured by an electronic device 30. The electronic device 30, acting as an image capturing unit, may employ various imaging sensors and interfaces. A color calibration module 503 is utilized to process the captured image and generate calibrated color intensity data. This color calibration module 503 can be implemented in software executed by the processing unit 502 (CPU or GPU) of the cloud server 50, or, for enhanced performance, may be partially or fully implemented within the electronic device 30 using a CPU, GPU, Digital Signal Processor (DSP), or even dedicated hardware like an Application-Specific Integrated Circuit (ASIC) or Field-Programmable Gate Array (FPGA). GPUs and DSPs are particularly well-suited for the parallel processing demands of image and color manipulation.

Once the relevant standard line and calibrated color intensity data are available, a normalization unit 5022 within the processing unit 502 performs the calculation to determine the concentration of the target analyte (e.g., gluten). For instance, the normalization unit 5022 leverages the computational capabilities of the processing unit 502, utilizing its Arithmetic Logic Unit (ALU) and Floating-Point Unit (FPU) for the necessary mathematical operations (division, multiplication, etc.). For large datasets or performance-critical applications, the normalization unit's functions may be accelerated by a GPU or a DSP. In highly optimized systems, dedicated hardware like an ASIC or FPGA could also be employed for the normalization unit.

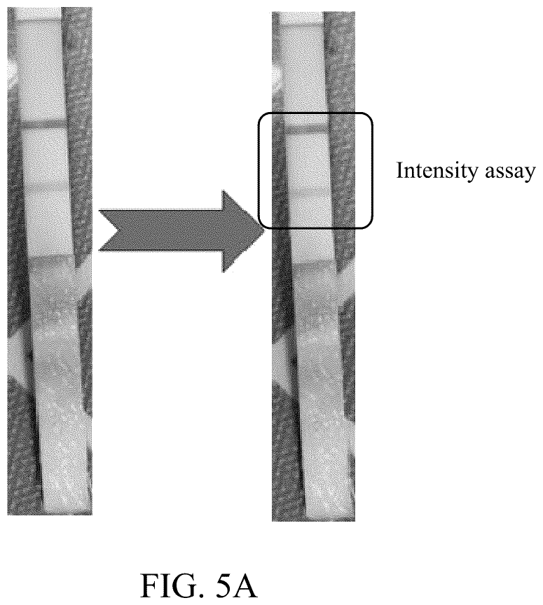

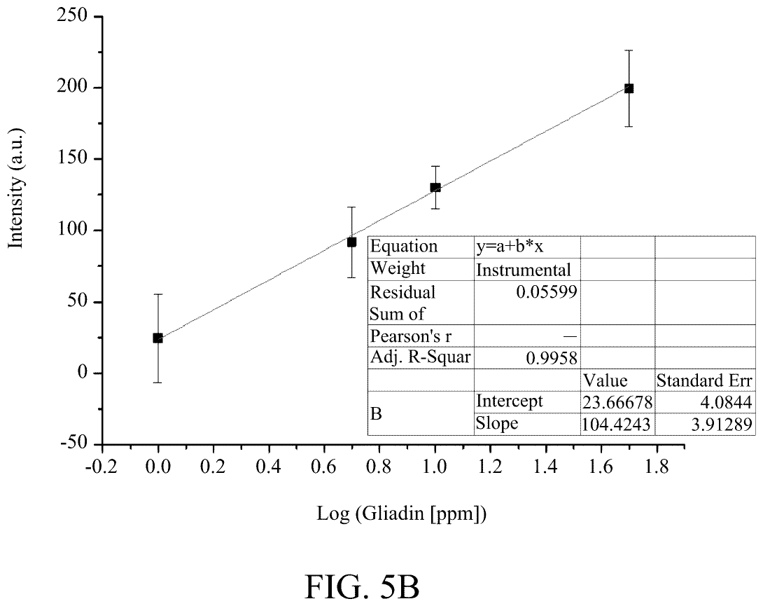

As shown in FIG. 5A, the image of the test line 104 and the control line 105 is converted to 8-bit grayscale by the electronic device 30 or other apparatus. FIG. 5B shows samples with varying doses of gliadin that were analyzed by the system and a response line that was generated.

Therefore, users can quantify gliadin concentrations through the system (including a smartphone-based image assay app). The compact design of the system allows for convenient use in various settings. According to the present disclosure, the detection process involves simple steps, and results are displayed in the electronic device 30.

### EXAMPLES

Various properties and efficacies will be illustrated by Examples below. The Examples set forth are used to illustrate the properties of the present disclosure which is not limited to those illustrated in the particular examples.

### Material

All solutions and samples were prepared using deionized water with a resistivity of 18.2 Ωcm⁻¹ from a Millipore Milli-Q water purification system (Millipore). Hydrogen tetrachloroaurate trihydrate (HAuCl4·3H2O), imidazole, propanol, methanesulfonyl chloride, gluten from wheat, and citric acid were procured from Sigma Aldrich. Tris buffer, PBS buffer, sodium bicarbonate, hydrochloride, sodium hydroxide, potassium chloride, sodium dihydrogen phosphate, sodium hydrogen phosphate, and sodium chloride were purchased from ACOS. Dichloromethane, acetonitrile, methanol, and ethanol were purchased from Tedia. Nitrocellulose membranes (Hi-Flow plus 120, Merck Millipore), sample pads (cellulose fiber, Merck Millipore), conjugate pads (cellulose fiber, Merck Millipore), absorbent pads, adhesive backing cards, and goat anti-mouse IgG were obtained from Rojan Azma Co (Tehran, Iran). Mouse Anti-Gliadin IgG was obtained from Chondrex Inc (Catalog No. 7148). Rabbir Anti-gliadin IgG was obtained from Merk (Catalog No. G9144). Trisodium citrate dihydrate, bovine serum albumin, Tween-20, phosphate buffer solution, PEG20000, glucose, and 0.22 μm filters were purchased from Merck.

### Statistical Analysis

All calculated data are presented as means±standard deviations. A p value of <0.05 was considered significant.

### Preparation Example 1: Synthesis of Ionic Liquid (IL)

An imidazolium-based IL was synthesized through an SN2 reaction with 1-methylsulfonic pentane. One equivalent (eq) of pentanol and 1.2 eq of triethylamine were added to a reaction bulb, followed by the addition of 30 mL of dichloromethane (DCM). The mixture was stirred in an ice bath for 5 min. Methanesulfonyl chloride (MsCl) at 1.1 eq was slowly added dropwise into the reaction bulb to form a mixture. Subsequently, the ice bath was removed, and the mixture was allowed to react at room temperature for 20 min to yield the initial reaction mixture.

The initial reaction mixture was then extracted three times with a 10% (w/v) aqueous solution of citric acid (water phase), followed by three instances of extraction with a 10% aqueous solution of sodium hydrogen carbonate (NaHCO₃) to produce an extract. After extraction, the solvent in the extract was removed by concentration under reduced pressure. 1,2-Dimethylimidazole at 0.9 eq and 50 mL of acetonitrile (ACN) were added, and the mixture was heated to 60° C. and maintained at this temperature for 12 h to generate a second reaction mixture. Subsequently, the solvent in the second reaction mixture was removed by concentration under reduced pressure to obtain the first crude product. This crude product was then extracted with hexane and dried by concentration under reduced pressure. The obtained IL had pentyl dimethyl imidazolium as the cation and methanesulfonate as the anion, i.e. [C5DMIM][OMs].

The nuclear magnetic resonance spectrum of [C5DMIM][OMs] (200 MHz, CDCl3) displayed signals at 0.86-0.88 (3H, t), 1.86-1.91 (4H, m), 2.85 (3H, s), 3.85-3.87 (2H, t), 3.91 (3H, s), 4.12 (3H, s), 7.84-7.85 (1H, d), and 7.87-7.88 (1H, s).

### Preparation Example 2: Preparation of Different Buffer Types

### 2-1. Preparation of PBS Buffer and PBS Buffer with 10% IL

I. PBS buffer: 800 mL of distilled water was provided in a Duran bottle. Next, 8 g of NaCl, 1.44 g of Na₂HPO₄, 0.2 g of KCl, and 0.24 g of KH₂PO₄ were added to the water. The pH was adjusted to 7.4 or 7.2 (depending on the application) by using HCl and add deionized water until the total volume reached 1 L, yielding the PBS buffer.
II. PBS buffer with 10% IL: 10 g of IL ([C5DMIM][OMs] prepared in preparation Example 1) was added to 990 mL of the PBS buffer to prepare a PBS buffer with 10% IL.

### 2-2. Preparation of Tris Buffer and Tris Buffer with 10% IL

I. Tris buffer: 800 mL of distilled water was provided in a suitable container, to which 121.14 g of Tris base was added and then the solution was adjusted to the desired pH by using HCl (typically around pH 7.0). Next, distilled water was added until the volume reached 1 L to obtain the Tris buffer.
II. Tris buffer with 10% IL: 10 g of IL ([C5DMIM][OMs] prepared in preparation Example 1) was added to 990 mL of the Tris buffer to prepare Tris buffer with 10% IL at room temperature.

### 2-3. Preparation of Carbonate Buffer and Carbonate Buffer with 10% IL

I. Carbonate buffer: 1.05 g of sodium bicarbonate and 9.274 g of sodium carbonate (anhydrous) were added to 800 mL of distilled water in a suitable container. Subsequently, distilled water was added to the solution until a total volume of 1 L was achieved, yielding the carbonate buffer.
II. Carbonate buffer with 10% IL: 10 g of IL ([C5DMIM][OMs] prepared in preparation Example 1) was added to 990 mL of the carbonate buffer in a suitable container to prepare a carbonate buffer with 10% IL.

### 2-4. Preparation of Citric Buffer and Citric Buffer with 10% IL

I. Citric buffer: 24.269 g of sodium citrate dihydrate and 3.358 g of citric acid were added to the solution to 800 mL of distilled water in a suitable container. The solution pH was adjusted to the desired level by using 0.1 N HCl (typical pH of approximately 6.0) as the citric buffer.
II. Citric buffer with 10% IL: 10 g of IL ([C5DMIM][OMs] prepared in preparation Example 1) and distilled water were added to 990 mL of the citric buffer in a suitable container until the total volume reached 1 L to prepare a citric buffer with 10% IL.

### Preparation Example 3: Preparation of AuNPs

AuNPs were synthesized using citric acid to reduce Au ions in a water solution. Specifically, 100 mL of HAuCl₄ (0.02%) was refluxed with constant stirring, after which 3 mL of 1% trisodium citrate solution was immediately added to the conical flask with continuous stirring. Within 2-5 min, the initial pale-yellow-colored solution turned colorless and changed to bluish-gray. After an additional 5 min, the solution turned reddish-purple, indicating the formation of AuNPs. The solution was stirred for another 10 min and then cooled to room temperature.

### Preparation Example 4: Preparation of a AuNP-Conjugated Antibody

Passive adsorption is a commonly used technique for conjugating antibodies to AuNPs. In this procedure, 100 μL of Rabbir Anti-gliadin IgG (G9144, Merk) solution (1 μg/mL) was added to an Eppendorf tube containing 1 mL of AuNPs. The mixture was rotated for 30 min to facilitate antibody adsorption. To block any remaining binding sites on the AuNP surface, 10% bovine serum albumin was added therein. Centrifugation (10000 rpm for 30 min at 4° C.) was used to separate the antibody-conjugated AuNPs from unbound reagents. This centrifugation step was repeated twice to ensure thorough washing. Finally, the obtained pellet was resuspended in 1% bovine serum albumin and stored at 4° C. until further use.

### Preparation Example 5: Preparation of a Lateral Flow Chip

The chip was constructed using a sample pad, conjugate pad, assay membrane (composed by nitrocellulose), absorbent pad, and adhesive backing cards. To form the test and control lines, Mouse Anti-Gliadin IgG (7148, Chondrex Inc) and goat anti-mouse IgG (100 μg/mL, Sigma Aldrich) were dispensed onto the assay membrane at a 6 mm distance. Sample pad preparation involved pH optimization and blocking buffer composition. The pad was treated with PBS containing 5% (w/v) bovine serum albumin, 0.5% Tween 20, 5% polyethylene glycol, and 0.05% (w/v) NaN3, followed by incubation for 30 min. The treated pad was then rinsed with PBS buffer and dried overnight at 37° C. The conjugate pad was prepared through soaking in the conjugate solution containing the AuNP-conjugated antibody prepared in the Preparation Example 4 and then incubated overnight at 37° C.

### Preparation Example 6: Preparation of Gliadin Standard Solution

Gliadin extraction from the flour samples was performed as follows: 1 g of flour was stirred with 10 mL of a 75% (v/v) ethanol solution to achieve a homogeneous mixture. The solution was then centrifuged at 6000 rpm for 10 min at room temperature, and the supernatant was subsequently diluted to 0.01, 0.1, 1, 5, 10, 20, and 40 ppm of gliadin standard solution using PBS buffer (measured by ELISA).

### Preparation Example 7: Preparation of Sample Fluid

### 7-1. Preparation of Specific Test Sample

Gluten was extracted from designated food samples (wheat, oat, corn, quinoa, rice, chickpea, chestnut, and almond flour) by using 75% (v/v) ethanol. The supernatant was filtered through a 0.22 μm filter and diluted with PBS buffer to create a 100 ppm gliadin standard solution. A series of gluten concentrations (0, 0.001, 0.1, 1, 5, 10, 20, and 40 ppm) were prepared by serially diluting the stock solution with PBS buffer.

### 7-2. Preparation of Real Samples

Food samples were collected from local supermarkets and restaurants. For each sample, the following procedure was used: 20 mg of food sample was combined with 1 mL of buffers prepared in the Preparation Example 2 and shaken three times to facilitate extraction

### Preparation Example 8: Preparation of Standard Line

To quantify gliadin concentration within the system, for each batch a response line was generated by testing the intensities of the test and control lines obtained from using samples with various given gliadin concentrations (FIG. 5B). The line was then incorporated into the iFAMs image assay system as the standard line for the batch, enabling quantitative analysis.

### Example 1: Characterization of AuNPs and AuNP-Conjugated Antibody

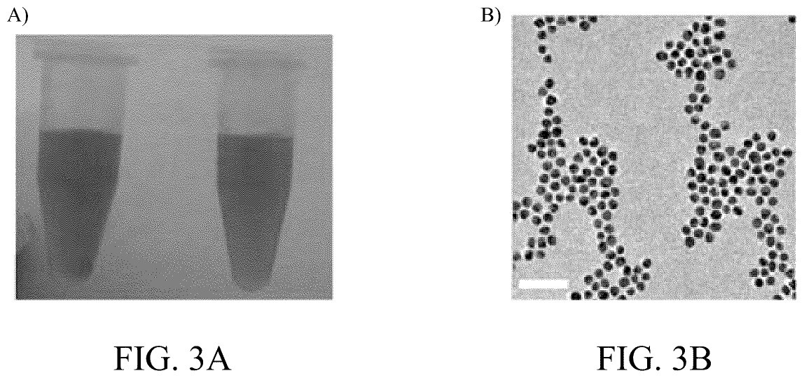

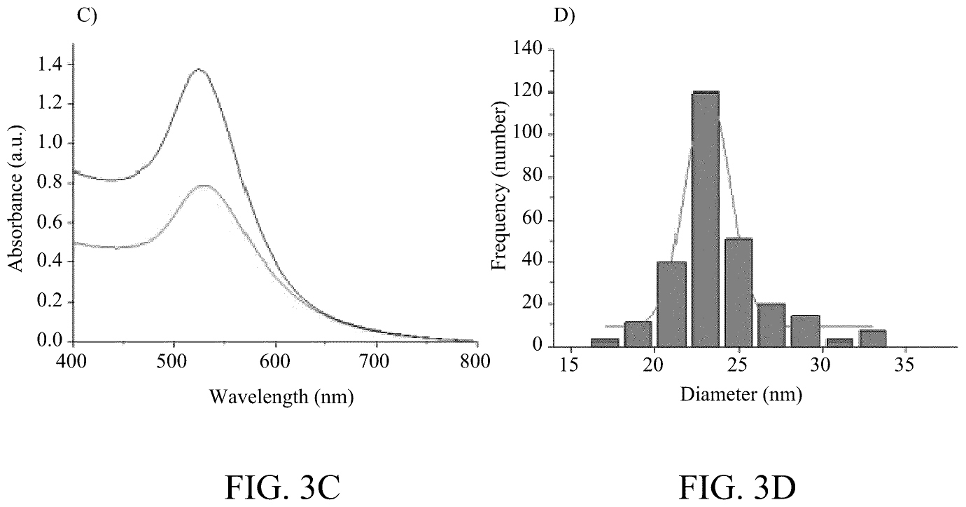

AuNPs exhibit a unique local surface plasmon resonance (LSPR) effect that is sensitive to particle size and surface chemistry. Accordingly, the proposed system of the present disclosure leverages this effect through the use of citrate-reduced AuNPs. AuNPs were prepared as described in the above Preparation Example 3, and the obtained solution of AuNP is shown in FIG. 3A (left panel). The AuNPs were then conjugated with the gliadin antibody to form the AuNP-conjugated antibody as described in the above Preparation Example 4, and the obtained solution of the AuNP-conjugated antibody is also shown in FIG. 3A (right panel). The results indicate that conjugation with the antibody did not engender a considerable color shift in the AuNPs. Furthermore, the size distribution and average diameter of the AuNPs were characterized using ultraviolet-visible (UV-VIS) light spectrophotometry and atomic force microscopy (AFM). As shown in FIG. 3C, the UV-VIS spectra revealed narrow absorption peaks for the unconjugated AuNPs (black line, 521 nm) and AuNP-conjugated antibody (red line, 523 nm). Moreover, the spherical morphology of the AuNPs was confirmed by the AFM results (FIG. 3B). ImageJ software was used to analyze the AFM images, and the results indicated a uniform size distribution with an average diameter of approximately 24 nm (FIG. 3D). Dynamic light scattering was also used to measure the size of the AuNPs. The hydrodynamic diameter of the unconjugated and antibody-conjugated AuNPs were 25 and 40 nm, respectively.

### Example 2: Optimal Antibody Concentrations for Conjugation with AuNPs

The pH value influences the conjugation of antibodies to AuNPs. To investigate the effect of pH on antibody-AuNP conjugation, successful conjugation was defined as the absence of notable color shifts upon antibody or salt addition. The results of the experiments have revealed that the optimal pH for conjugation was 8. At this pH, the highest stable concentration of the antibody-conjugated AuNPs in solution was achieved when the antibody concentration was 1 μg/mL. This was determined by observing color changes in AuNP solutions containing different antibody concentrations in the presence of NaCl. Moreover, these results were validated by measuring and comparing the UV-VIS absorption spectra of the synthesized AuNPs. Owing to the sensitivity and unique properties of LSPR in AuNPs, changes in absorbance spectra can indicate surface binding events. In the present disclosure, when the antibody concentration was 1 μg/mL, the absorption peak at 523 nm increased and exhibited a shift toward longer wavelengths. This spectral change confirms the interaction between antibodies and the AuNP surface. Therefore, the AuNP-conjugated antibody used in the following Examples was prepared by using antibody at a concentration of 1 μg/mL.

### Example 3: Integrated Food Allergy and Microorganism Sensor (iFAMs) System

Personal dietary intake can be tracked and gluten data with timestamps in a cloud server can be recorded by means of the iFAMs system of the present disclosure with a smartphone app. Specifically, about 20 mg of the sample was added into a container comprising the formulation of the present disclosure and the cover of the container was closed before shaking three times. Subsequently, the small cover of the container was opened slightly, and some solution was dropped into the sample pad of the chip. The test result would appear on the detecting area 103 in the result window after 1.5 min. Subsequently, an image of the chip was captured by an electronic device and then updated to the cloud server. In the cloud server, the image of the test line and the control line was converted to 8-bit grayscale by using IMAGE J program, and by means of a color calibration module, a color reference mark on the lateral flow chip was calibrated, and colors of the test line and the control line are simultaneously calibrated. In the same time, the batch information recoded in an identification code on the chip was identified by the cloud server, and then the gliadin concentration of the sample was normalized and calculated by means of a standard line prepared in the Preparation Example 8 for that batch by the cloud server. The gliadin content was then transmitted from the cloud server back to the electronic device and displayed by a display module of the electronic device. The results displayed in the display module in about 15 s.

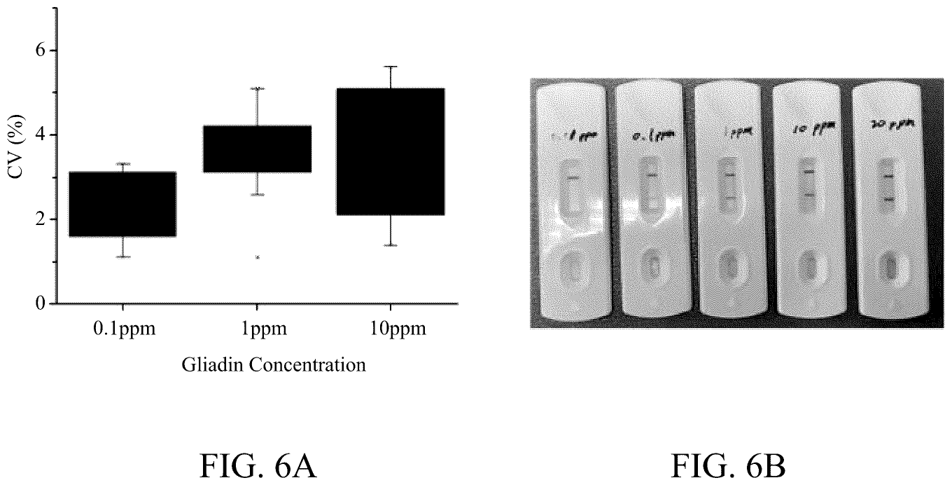

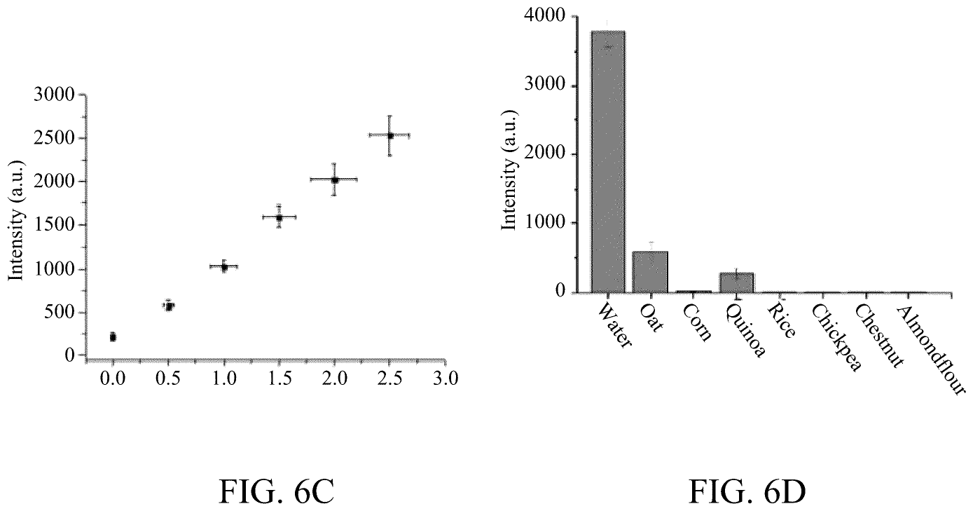

To evaluate the detecting precision of the iFAMs system, intra-assay variations were assessed by measuring eight replicates of three different standard concentrations (0.1, 1, and 10 ppm). The results revealed excellent intra-assay precision, with variations below 6% (FIG. 6A). Furthermore, inter assay variations were evaluated and found to be less than 7%. For comparative purposes, the same samples were analyzed using the conventional ELISA technique. The iFAMs system results exhibited a strong correlation with the ELISA results (R²=0.995). Notably, the iFAMs system offered a considerable advantage in terms of speed, delivering results within 2 min, when compared with the ELISA technique, which required a 3-h analysis time.

Further, to evaluate the sensitivity of the iFAMs system, eight gluten concentrations (0.001, 0.01, 0.1, 1, 5, 10, 15, and 20 ppm) were tested in triplicate. A visible pink color appeared along the test line as the gluten concentration was increased from 0.01 to 20 ppm (FIG. 6B). Furthermore, the color intensity of the test line increased with the gluten concentration (FIG. 6C).

The following equation was used to calculate the detection limit of the iFAMs: Sdl=Sreag+3σreag, where Sdl is the detection limit, Sreag is the signal of the reagent blank, and σreag is the standard deviation of the reagent blank. The calculation indicated the detection limit to be 0.04 ppm, which is lower than the eliciting dose thresholds for gluten allergens (20 ppm).

Moreover, specificity is a crucial metric for biosensors. To evaluate the specificity of the proposed iFAMs system, flour samples derived from various cereals were tested: wheat, oat, corn, quinoa, rice, chickpea, chestnut, and almond. A red line on the test strip only observed for the gluten-containing cereals: wheat, oat, and quinoa. Moreover, the red line for wheat flour appeared within 2 min, likely owing to its high gluten concentration (FIG. 6D). Each sample was tested six times under the same experimental conditions. Despite being intrinsically gluten-free, both oat and quinoa showed traces of gluten in this analysis. This finding is likely a result of cross-contamination, a common problem with “high-risk” gluten-free grains. Cross-contact often occurs during growing, harvesting, or processing alongside gluten-containing grains such as wheat, barley, and rye.

As shown in the above results, the iFAMs system of the present disclosure can precisely and rapidly test the gliadin concentration. When using the iFAMs system for gliadin testing, a positive test result, signified by the appearance of both test and control lines on the assay membrane, is considered to indicate the presence of ≥0.1 ppm of gluten in the sample; and a negative test result, signified by the appearance of only the control line, is considered to indicate a gluten concentration of <0.01 ppm.

The time and location of the test can also be recorded when using the iFAMs system, and thus the cloud server can document the results alongside local restaurant information. These data can be subsequently used to generate an evidence-base restaurant map, which can be shared online.

### Example 4: Gliadin Solubility in Different Buffer Systems

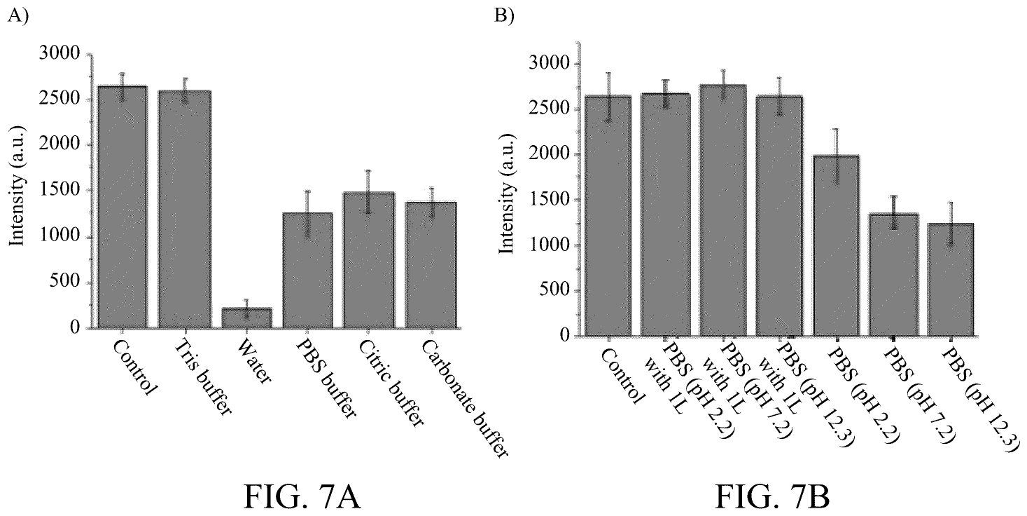

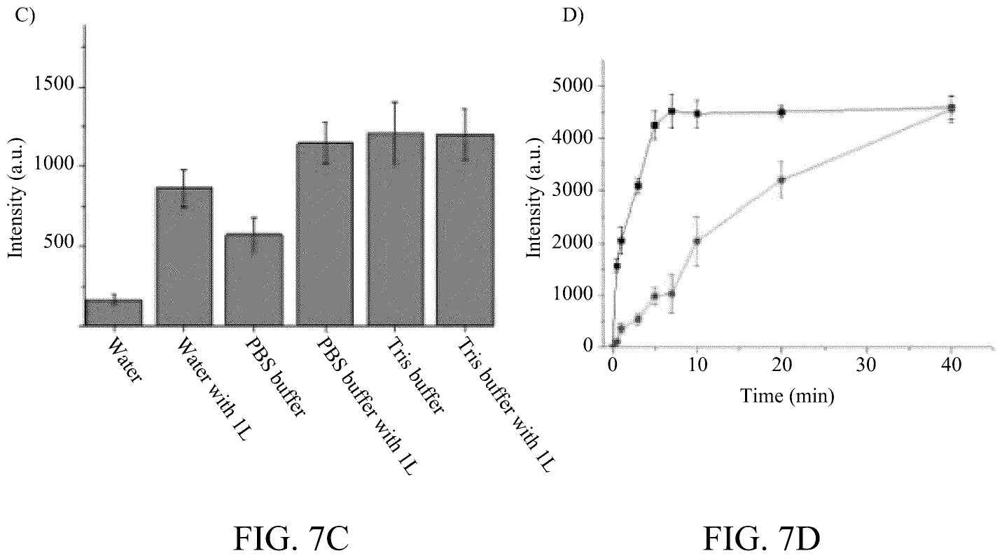

To investigate gliadin solubility in different buffer solutions, 3 g of gluten (excess amount, sourced from Sigma Aldrich) was added to 10 mL of various buffer solutions prepared in the Preparation Example 2 (i.e., Tris buffer, PBS buffer, citric buffer and carbonate buffer) and water. The mixtures were homogenized by stirring for 30 min and allowed to rest for 5 min. After the extraction process, all samples were centrifuged at 8,500 rpm for 3 min to remove any particulates. The supernatant solutions were then passed through a 0.22-μm filter to obtain clarified solutions for further analysis. Ethanol solution (75 wt %) was used as the control. To ensure accurate comparison with the other samples, the control solution was diluted five times with deionized water prior to analysis. The iFAMs system was used to quantify the gliadin concentration in all clarified samples (FIG. 7A).

As shown in FIG. 6A, the gliadin solubility levels were highest in alcohol and Tris buffer and poor in water alone. Moreover, PBS buffer, citric buffer, and carbonate buffer could also be used to dissolve gliadin. These findings indicate that salts can increase gliadin solubility in water.

### Example 5: Gliadin Solubility in pH Buffer with and without IL

To investigate the effects of pH and IL on gliadin solubility, 3 g of gluten (excess amount, sourced from Sigma Aldrich) was added to 10 mL of pH-adjusted (pH=2.2, 7.2, and 12.3, adjusted by using HCl or NaOH) PBS buffer with and without 10% of IL as prepared in Preparation Example 2. The mixtures were homogenized by stirring for 30 min and then allowed to stand for 5 min. After centrifugation at 8500 rpm for 3 min and filtration through a 0.22-μm filter, the iFAMs system of the present disclosure was used to quantify the gliadin concentration (FIG. 7B). A 75% alcohol solution (diluted five times with deionized water) served as the control.

As expected, acidic pH enhanced gliadin solubility, particularly in PBS buffer. However, the addition of IL considerably increased gliadin solubility across all tested pH values in PBS, demonstrating the positive effect of IL on extraction efficiency.

### Example 6: Environmental Effect

Ionic strength and pH can influence antibody binding affinity in some cases. Accordingly, the potential effect of ionic strength and pH on antibody binding affinity was investigated. To isolate the effects of different buffer solutions, gluten standard solution was prepared by using 75% alcohol and diluted 50 times in PBS buffer. Then this standard solution (1 mL) was diluted with various buffer solutions (4 mL) containing water, PBS, or Tris (with and without IL) as prepared in the Preparation Example 2 to minimize the influence of alcohol on antibody binding, the iFAMs system of the present disclosure was used to quantify the gliadin concentration (FIG. 7C).

The results shown in FIG. 7C demonstrate a considerable difference in signal intensity between water with and without IL. Water alone exhibited a low signal intensity level, likely owing to gliadin precipitation. However, the addition of IL considerably enhanced the signal intensity level, suggesting that IL enhances gliadin solubility in water. A similar, although less pronounced, effect was observed in PBS buffer with and without IL. The presence of IL did not considerably alter the signal intensity in the Tris buffer solution at the end point of this Example (this may be because the extraction was conducted for a long time).

### Example 7: Time Dependency of Gliadin Solubility in Tris Buffer with and Without IL

IL shows promise as solvents for organic transformations. Therefore, the effect of IL on gliadin solubility in Tris buffer over time was further investigated.

The effect of extraction time on gliadin solubility in Tris buffer and Tris buffer with 1% [C5DMIM][OMs]_{aq} was investigated. For each buffer, 3 g of gliadin was added to separate sample vials and homogenized the mixtures by shaking. The mixtures were then allowed to rest for various time intervals (0.5, 1, 3, 5, 7, 10, 20, and 40 min). Precipitates were removed using a 0.22 μm filter, and gliadin solubility was determined using a lateral flow assay. Results were quantified using an image analysis software tool (color intensity assay on website).

As a control, the experiment using PBS buffer in place of [C5DMIM][OMs]_{aq} was repeated, following the same extraction procedure and analysis methods. The results are shown in FIG. 7D.

As illustrated in FIG. 7D, gliadin solubility in Tris buffer alone gradually increased with time, with the solubility level peaking after 40 min at room temperature (red cycle). Moreover, the addition of 1% IL considerably enhanced gliadin solubility, with maximum solubility occurring within just 5 min (black square). The results demonstrate that the addition of the IL ([C5DMIM][OMs]_{aq}) considerably improved gliadin extraction efficiency in Tris buffer. The extraction effectiveness observed with IL was more than eight times greater than that with Tris buffer alone. This finding demonstrates the ability of IL to enhance gliadin solubility. For gluten detection, effective extraction directly influences the sensitivity of assays. Therefore, the use of Tris buffer with IL affords a rapid and highly efficient method for gliadin extraction, potentially improving the sensitivity of gluten tests.

### Example 8: Gliadin Recovery Rate with the iFAMs System

Recovery rate is a crucial metric for evaluating extraction systems; this is because it reflects the effectiveness of extracting a target substance from a sample. In the present disclosure, the gliadin recovery performance of the iFAMs system was assessed.

Hydrophobic proteins are traditionally extracted using alcohol solutions. In this example, a 75 wt % ethanol solution was prepared. Subsequently, 3 g of bread flour (Blue Jacket Strong Flour, Lien Hwa Milling) was mixed with 10 mL of 75 wt % ethanol solution, followed by extraction at room temperature for 5 min. The mixture was centrifuged (8500 rpm, 3 min) and filtered (0.22 μm pore size) to obtain a clear gliadin extract. The gliadin concentration was determined using a wheat/gluten (Gliadin) ELISA kit (Crystal Chem, AOAC no. 011804). Samples were diluted by at least 50-fold to ensure accurate quantification. The gliadin concentrations in both 75 wt % ethanol extraction groups exceeded 1700 ppm.

The protocol for using the ELISA kit was as follow: Initially, 1 g of the homogenized mixture was suspended in 10 mL of 40% ethanol. Subsequently, the suspension was mixed for an additional 5 min to ensure thorough homogeneity. The samples were then centrifuged for 10 min at 2500 g. The resulting particle-free solution was diluted at 1:50 in 1× diluent buffer. All materials were brought to room temperature (20-25° C.) before use. The standards and samples were assayed in duplicates. Specifically, 100 μL of samples or standards was added to the antibody-coated microplate and incubated the plate at room temperature for 20 min. Each well was aspirated and washed three times with 1× wash buffer (300 μL) by using a squirt bottle or manifold dispenser. The remaining liquid was completely removed after each wash to ensure optimal performance. 100 μL of the antibody conjugate was added to each well and then incubated the plate at room temperature for 20 min on a microplate shaker. Each well was aspirated, and the washing process was repeated. The TMB substrate was brought to RT, and 100 μL of the substrate was added to each well (including blank wells). The plate was incubated for 20 min at room temperature in the dark. 100 μL of stop solution was then added to each well (including blank wells). Optical density was immediately measured with a microplate reader at 450 nm.

In addition, rice noodles constitute a well-known gluten-free food and were selected as the sample for the recovery test. 40 g of dried gluten-free rice noodles (Organic Rice Noodles, Yuan Shun Food) was rehydrated in water. After draining, the noodles were soaked in 10 mL of a 10-ppm gliadin solution (prepared in 100% ethanol) at room temperature. Owing to their high specific surface area, the rice noodles effectively adsorbed the gliadin. After the ethanol solvent was evaporated, the gliadin-spiked rice noodles were lyophilized. The lyophilized gliadin-spiked rice noodles were analyzed using the iFAMs system at room temperature to assess gliadin recovery. The resulting data are listed in the following Table 1.

TABLE 1

| | Standard | Extraction | |
|---|---|---|---|
| | gluten | gliadin | Recovery |
| [symbol] | (ppm) [symbol] | (ppm) [symbol] | (%)¹ [symbol] |
| 1 [symbol] | 10 [symbol] | 9.3 [symbol] | 93% [symbol] |
| 2 [symbol] | 10 [symbol] | 9.6 [symbol] | 96% [symbol] |
| 3 [symbol] | 10 [symbol] | 10.5 [symbol] | 105% [symbol] |
| 4 [symbol] | 10 [symbol] | 9.4 [symbol] | 94% [symbol] |
| 5 [symbol] | 10 [symbol] | 10.1 [symbol] | 101% [symbol] |
| Average [symbol] | 10 [symbol] | 9.78 [symbol] | 97.8% [symbol] |

A 100% recovery rate was defined as a recovery of 200 ppm of gliadin. Across five repetitions of the recovery test using the iFAMs system, the average recovery rate achieved by the iFAMs system using Tris buffer with 1% of IL was determined to be 97.8%.

### Example 9: Comparing the System of the Present Disclosure with Commercially Available Products

The system of the present disclosure is a rapid and user-friendly assay method. This Example compares the iFAMs system of the present disclosure with commercially available products, such as the R-Biopharm gliadin test, which is the only strip-based assay for gliadin detection using the G5 antibody. The sensitivity and specificity of the G5 antibody contribute to the high sensitivity of the R-Biopharm gliadin test. However, the pretreatment process in the R-Biopharm gliadin test still requires sample heating and is time-consuming. Nevertheless, the R-Biopharm gliadin test remains widely used for gliadin detection.

The results of comparing the performance of the iFAMs system with that of the R-Biopharm gliadin test in processing food samples (with or without being labeled as food containing gluten) are listed in the following Table 2.

TABLE 2

| | Label | | | R-Biopharm |
|---|---|---|---|---|
| | GF | IFAMs | | (R7003) |
| Guerrero Tostatas | Yes | 0 | ppm | <5 ppm |
| Krusteaz GF All-purpose flour | Yes | 0 | ppm | <5 ppm |
| Krusteaz GF Honey cornbread mix | Yes | 0.4 | ppm | <5 ppm |
| Sprouts GF Steel cut Oats | Yes | 0 | ppm | <5 ppm |
| Pillsbury GF Choc Fudge brownie | Yes | 0 | ppm | <5 ppm |
| Mix | | | | |
| Lay's Stax Mesquite Barbecue | Yes | 0 | ppm | <5 ppm |
| Chips ChipsMcCormic | | | | |
| Kelloggs Multi grain club crackers | No | >40 | ppm | >40 ppm |
| Smarties candy Bracelate | No | 0 | ppm | <5 ppm |
| Fritos Chili Cheese Chips | No | 0 | ppm | <5 ppm |
| Mild taco seasoning | No | 0 | ppm | <5 ppm |
| Cap't Crunch Berries | No | 0.6 | ppm | <5 ppm |

The results shown in Table 2 revealed a high level of consistency between the iFAMs system and R-Biopharm gliadin test. This demonstrates that the iFAMs system is effective in detecting gliadin during food processing.

The performance of the iFAMs system was further evaluated by testing various consumer food products, including packaged staples and desserts. Furthermore, samples with fermentation (beer and sauce) were also tested. Small samples (40 mg) were collected, and solid foods were crumbled before analysis. The performance of the iFAMs system was compared with that of ELISA and the Ecove gluten sensor, another rapid test with one-pot extraction technology. Ecove claims higher sensitivity (1 ppm) than ELISA. The results are shown in the following Table 3.

TABLE 3

| | iFAMs | | ELISA | | Ecove |
|---|---|---|---|---|---|
| Ferrero | 7 | ppm | 8 | ppm | N.D. |
| Bagel | >40 | ppm | >40 | ppm | Yes |
| Fried dumplings | >40 | ppm | >40 | ppm | N.D. |
| Toast | >40 | ppm | >40 | ppm | N.D. |
| Lay's Stax Original Potato Crisps | 0.5 | ppm | 0 | ppm | N.D. |
| Nestle Nesquik Chocolate Syrup | 0.6 | ppm | 0 | ppm | N.D. |
| M & M's Crispy Milk Chocolate Bar | 1.8 | ppm | 2.2 | ppm | N.D. |
| Campbell's Chunky New England Clam Chowder | 7.6 | ppm | 6.4 | ppm | Yes |
| Lindt Swiss Classic Milk Chocolate | 3.2 | ppm | 2.1 | ppm | N.D. |
| S & B Golden Curry Sauce with Vegetables Mild | 10.2 | ppm | 9.8 | ppm | Yes |
| Hershey's Cookies ‘N’ Crème Candy Bar | 7.6 | ppm | 9.4 | ppm | Yes |
| Nestle Kit Kat Chunky Peanut Butter Chocolate | >40 | ppm | >40 | ppm | Yes |
| Barilla Capellini n.1 | >40 | ppm | >40 | ppm | N.D. |
| Lotus Biscoff Spread Crunchy | >40 | ppm | >40 | ppm | Yes |
| Munchy's Oat Krunch Crackers Strawberry & Blackcurrant | >40 | ppm | >40 | ppm | Yes |
| Bento Squid Seafood Snack Original Thai Chili Sauce | >40 | ppm | >40 | ppm | Yes |
| Big Lost: Crazy Woman | 0 | ppm | 0 | ppm | N.D. |
| Big List: Wild Man | 0 | ppm | 0 | ppm | N.D. |
| Pine Ridge: Barbecue & Dipping Sauce | 0 | ppm | 0 | ppm | N.D. |
| Pine Ridge: Jalapeno Barbecue & Dipping Sauce | 0 | ppm | 0 | ppm | N.D. |
| Pine Ridge: Sweet Mustard Sauce | 0 | ppm | 0 | ppm | N.D. |
| Jackson Hole Still Works: Great Grey Gin | 0 | ppm | 0 | ppm | N.D. |
| Jackson Hole Still Works: Vodka | 0 | ppm | 0 | ppm | N.D. |
| Jackson Hole Still Works: Absaroka Double Cask Gin 49 | 0 | ppm | 0 | ppm | N.D. |
| Budweiser beer | 12.6 | ppm | 11.4 | ppm | Yes |
| Lee Kum Kee Premium Oyster Flavored Sauce | 3.1 | ppm | 2.4 | ppm | N.D. |
| Toblerone Swiss Milk Chocolate with Honey and Almond Nougat | 0.4 | ppm | 0 | ppm | N.D. |

As shown in the Table 3, the results revealed consistency between the iFAMs system and ELISA. In some cases, where the gliadin concentration was below the sensitivity threshold of ELISA (2 ppm), detection may not occur. Overall, the iFAMs system and ELISA exhibited a high level of consistency. However, the performance of the Ecove gluten sensor was not satisfactory, particularly evident in its inability to detect gliadin in toast and noodle samples. Even after three repeated tests on the noodles and toast, Ecove failed to provide a clear positive result.

It can be seen from the examples above, the formulation of the present disclosure (comprising Tris buffer and ionic liquid) can rapidly and effectively extract water-insoluble proteins such as gliadin. Moreover, the kit comprising the lateral flow chip and the aforementioned formulation can be used to detect the gliadin in the material (e.g., food), and the amount of the gliadin can be further quantified by the iFAMs system provided by the present disclosure. This system can help individuals make informed decisions or choices and can eliminate unnecessary dietary restrictions. Moreover, the iFAMs system is compact, rapid, user-friendly, and quantitative. It is also cost-effective, with assay costs under $5 per antigen and no additional equipment requirements. The iFAMs system is highly adaptable and outperforms other consumer gluten detection methods because it eliminates complicated pretreatment steps and multi-solvent requirements. Its size and ease of use enable widespread application in various settings, including safeguarding consumer health, quality control, environmental monitoring, and supply chain oversight.

Compared with other rapid tests and ELISA techniques, the iFAMs system offers advantages such as ease of use, speed, accessibility, and broad applicability. Furthermore, the limit of detection (LOD) of the iFAMs system (0.04 ppm) is also lower than other lateral flow assay known in the art (5 ppm for 3M gluten protein rapid kit; 3 ppm for GlutenTox Sticks; and 10 ppm for EZ gluten). These features make it a promising tool for various food safety applications.

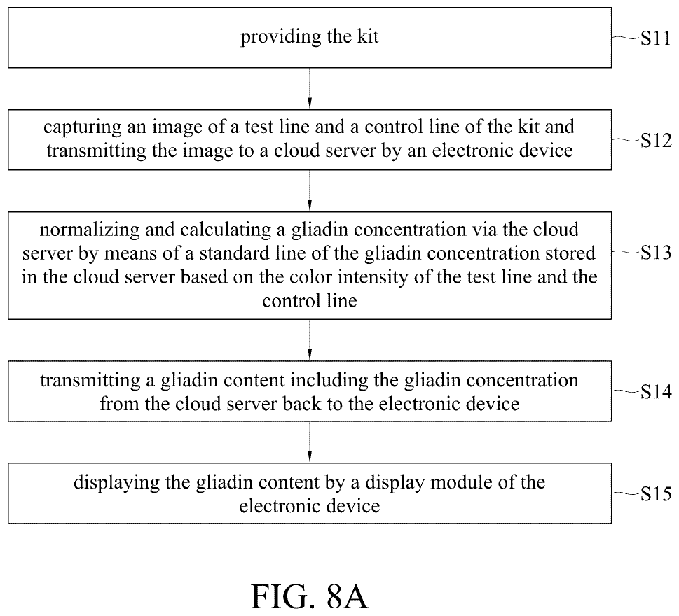

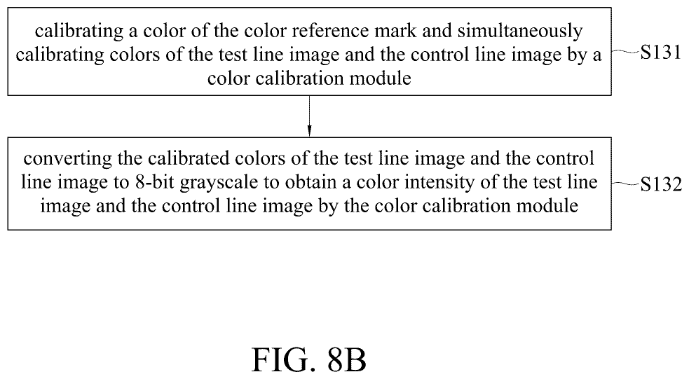

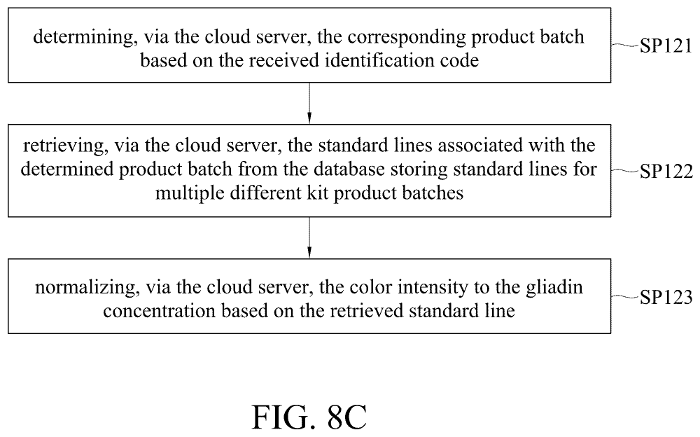

FIG. 8A and FIG. 8B are flow charts showing steps of a method for detecting a food allergen according to the present disclosure, and FIG. 8A and FIG. 8B are illustrated in conjunction with the description of the aforementioned embodiment. The method at least includes the following steps S11 to S15.

In step S11, the kit is provided.

In step S12, an image of a test line and a control line formed in the detecting area of the kit is captured, and the image is transmitted to a cloud server by an electronic device, and wherein the image includes the detecting area, the identification code, and the color reference mark of the kit.

In step S13, a gliadin concentration is normalized and calculated via the cloud server by means of a standard line of the gliadin concentration stored in the cloud server based on the color intensity of the detecting area in the image received via a data transmission module of the cloud server.

In step S14, a gliadin content including the gliadin concentration is transmitted from the cloud server back to the electronic device via the data transmission module. In step S15, the gliadin content is displayed by a display module 301 of the electronic device. In one embodiment, the display module 301 may be implemented as a Liquid Crystal Display (LCD) or an Organic Light Emitting Diode (OLED).

In addition, the method further includes steps S131 and S132 while executing step S13.

In one embodiment, when normalizing and calculating a gliadin concentration based on the color intensity of the detecting area in the image, the method further comprises step S131, calibrating a color of the color reference mark and simultaneously calibrating colors of the test line image and the control line image by a color calibration module of the cloud server; and step S132, converting the calibrated colors of the test line image and the control line image to 8-bit grayscale to obtain a color intensity of the test line image and the control line image by the color calibration module of the cloud server.

In one embodiment, the gliadin content with timestamp and location is stored in a database. For example, the database is a cloud database.

In one embodiment as illustrated in FIG. 8C, the database storing a plurality of standard lines for multiple different kit product batches, and the method further includes steps SP121 to SP123 before executing step S13:
SP121, determining, via the cloud server, the corresponding product batch based on the received identification code;
SP122, retrieving, via the cloud server, the standard lines associated with the determined product batch from the database storing standard lines for multiple different kit product batches; and
SP123, normalizing, via the cloud server, the color intensity to the gliadin concentration based on the retrieved standard line.

Finally, in one embodiment of the present disclosure, a computer program product is provided and utilizes the app, firmware, or cloud technology of the aforementioned system to execute the aforementioned method, and the computer program product can automatically store the gliadin content with timestamp and location in the cloud server.

While some of the embodiments of the present disclosure have been described in detail above, it is, however, possible for those of ordinary skill in the art to make various modifications and changes to the particular embodiments shown without substantially departing from the teaching and advantages of the present disclosure. Such modifications and changes are encompassed in the scope of the present disclosure as set forth in the appended claims.

What is claimed is:

1. A formulation for extracting a water-insoluble protein, comprising a Tris buffer and an ionic liquid.

2. The formulation of claim 1, wherein the ionic liquid is comprised in the formulation at a concentration of 0.05-1.5 wt %, based on the total weight of the formulation.

3. The formulation of claim 1, wherein the ionic liquid is an imidazolium-based ionic liquid comprising an imidazolium cation and an organic or inorganic anion, wherein the imidazolium cation has a structure of formula (I): 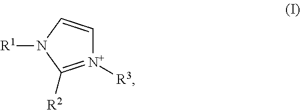
wherein R¹ is H or methyl, R² is H or methyl, and R³ is absent or an alkyl group having 3 to 12 carbon atoms,
and wherein the anion is selected from the group consisting of methanesulfonate (MSO), Cl⁻, F⁻, NO₃⁻, HSO₄⁻ and H₂PO₄⁻.

4. The formulation of claim 1, wherein the imidazolium cation is pentyl dimethyl imidazolium, heptyl dimethyl imidazolium, nonyl dimethyl imidazolium or dodecyl dimethyl imidazolium, and the anion is methanesulfonate, NO₃⁻, HSO₄⁻ or H₂PO₄⁻.

5. The formulation of claim 1, wherein the water-insoluble protein is gliadin.

6. The formulation of claim 1, which has a pH value ranges from 1 to 6.

7. A kit for detecting gluten, comprising:
a lateral flow chip, which comprises
a backing card;
an assay membrane disposed on the baking card, wherein the assay membrane has a detecting area with a first anti-gliadin antibody immobilized for forming a test line, and a secondary antibody against a AuNP-conjugated antibody immobilized for forming a control line, wherein the AuNP-conjugated antibody is formed by conjugating a second anti-gliadin antibody and an Au nanoparticle, and the control line being arranged staggered position relative to the test line;
a conjugate pad disposed upstream of the assay membrane, wherein the conjugate pad is adjacent to or partially covers the assay membrane, and at least part of the conjugate pad is coated with the AuNP-conjugated antibody;
an absorbent pad disposed downstream of the assay membrane, wherein the absorbent pad is adjacent to or partially covers the assay membrane, with a space between the conjugate pad and the absorbent pad to expose the detecting area of the assay membrane; and
a sample pad disposed upstream of the conjugate pad, wherein the sample pad is adjacent to or partially covers the conjugate pad; and
a container, which comprises the formulation of claim 1.

8. The kit of claim 7, wherein a material forming the assay membrane is selected from at least one of the group consisting of nitrocellulose, polyvinylidene difluoride (PVDF) and cellulose acetate.

9. The kit of claim 7, wherein the first anti-gliadin antibody and the second anti-gliadin antibody are different from each other and are independently selected from monoclonal antibody, polyclonal antibody and recombinant antibody.

10. The kit of claim 9, wherein each of the first anti-gliadin antibody and the second anti-gliadin antibody is selected from mouse anti-gliadin antibody, rabbit anti-gliadin antibody and recombinant human anti-gliadin antibody.

11. The kit of claim 7, wherein the kit further comprises an identification code.

12. The kit of claim 11, wherein the identification code is a 1D Barcode and/or a 2D Barcode.

13. The kit of claim 11, wherein the kit further comprises a color reference mark on the lateral flow chip.

14. The kit of claim 7, further contains a sampling tool for sampling the material to be tested.

15. The kit of claim 7, which further contains an operation instruction.

16. A method for detecting a food allergen, comprising:
receiving an image via a data transmission module of a cloud server, wherein the image includes the detecting area, the identification code, and the color reference mark of the kit of claim 13, and wherein the image is captured by an electronic device and transmitted to the cloud server;
normalizing and calculating a gliadin concentration via the cloud server by utilizing a standard line of gliadin concentration stored in the cloud server based on the color intensity of the detecting area in the image; and
transmitting a calculated gliadin content including the gliadin concentration from the cloud server back to the electronic device via the data transmission module.

17. The method of claim 16, wherein the image includes a test line image and a control line image formed in the detecting area, and the method further comprising:
calibrating a color of the color reference mark and simultaneously calibrating colors of the test line image and the control line image by a color calibration module of the cloud server; and converting the calibrated colors of the test line image and the control line image to 8-bit grayscale to obtain a color intensity of the test line image and the control line image by the color calibration module of the cloud server.

18. The method of claim 16, wherein the gliadin content with timestamp and location is stored in a database.

19. The method of claim 18, wherein the database is a cloud database.

20. The method of claim 19, wherein the database storing a plurality of standard lines for multiple different kit product batches, and the method further comprising:
determining, via the cloud server, the corresponding product batch based on the received identification code;
retrieving, via the cloud server, the standard lines associated with the determined product batch from the database storing standard lines for multiple different kit product batches; and
normalizing, via the cloud server, the color intensity to the gliadin concentration based on the retrieved standard line.
